(19) United States
(12) Patent Application Publication
(10) Pub. No.: US 2004/0259090 A1
(43) Pub. Date: Dec. 23, 2004

(54) Use of a biochip for the diagnosis of sepsis or sepsis related syndrome

(76) Inventors: Peter Franz Zipfel, Jena (DE); Hans-Peter Saluz, Oberbodnitz (DE); Stefan Russwurm, Jena (DE); Konrad Reinhart, Jena (DE)

Correspondence Address: Douglas J Christensen, Patterson Thuente Skaar & Christensen, 4800 IDS Center, 80 South 8th Street, Minneapolis, MN, 55402, US

(21) Appl. No.: 10/482,353

(22) PCT Filed: Jun. 28, 2002
(86) PCT No.: PCT/EP02/07179

(30) Foreign Application Priority Data
Jun. 29, 2001 (EP) 01115946.4

(51) Int. Cl. C12Q 1/68; G01N 33/53; C12M 1/34

(52) U.S. Cl. 435/6; 435/7.1; 435/287.2

(57) ABSTRACT

The application relates to the use of a nucleic acid and/or protein chip comprising probe nucleic acids and/or probe proteins, which are specific for cellular stress, inflammatory and immune reactions, associated with stress, inflammatory and immune reactions, induced during acute phase responses or any combination thereof and which are immobilised on a carrier for the diagnosis of sepsis or sepsis related syndrome.

### CROSS-REFERENCE TO RELATED APPLICATIONS

This application claims priority to PCT Application Serial No. PCT/EP02/07179, designating the United States, filed on Jun. 28, 2002, incorporated herein by reference, which claims priority to European Application No. 01 115 946.4, incorporated herein by reference.

### FIELD OF THE INVENTION

The invention relates to the use of a nucleic acid and/or a protein chip for the diagnosis of sepsis or sepsis related syndrome.

### BACKGROUND OF THE INVENTION

Sepsis and its sequalae of septic shock and multi-organ failure are the most common causes of death in operative intensive care units.

Sepsis—also commonly referred to as blood poisoning—is the most aggressive form of an infection. Basically, any infection can lead to sepsis if the body is not able to limit the infection to its place of origin. Then both the components of the bacteria as well as andogenous mediators can damage various organs, even far from the site of the infection. Within only a few hours a life-threatening condition with multiple organ failure can develop. The 40% to 60% mortality rate for sepsis has remained unchanged over the past decades. Approximately 5%-10% of all hospital patients become ill with an infection. In Germany alone, such infections lead to sepsis in 80,000 patients per year.

Leading international researchers claim that the main reason for the failure of new approaches in treatment lies often in a delayed and unspecific diagnosis of sepsis. An earlier and more effective treatment will be possible only through early diagnosis and an improved characterization of a patient's host response.

It is in particular necessary that the diagnosis of sepsis and also sepsis related syndrome can be carried out very quickly in order to start with the appropriate therapy soon and, furthermore, it is necessary to analyse a huge number of samples from patients in a very short time.

Thus, the technical problem underlying the present invention is to diagnose sepsis and sepsis related with the aforementioned advantages.

### SUMMARY OF THE INVENTION

This technical problem is solved by the use of a nucleic acid and/or a protein chip (biochip) comprising probe nucleic acids and/or probe proteins, which are specific for cellular stress, inflammatory and immune reactions, associated with stress, inflammatory and immune reactions, induced during acute phase responses or any combination thereof and which are immobilised on a carrier, in particular in an orderly grid pattern, for the diagnosis of sepsis or sepsis related syndrome.

According to the present invention, the term “diagnosis” does also comprise the monitoring of the course, the detection of the severity and the determination of the individual prognosis each of sepsis and sepsis related syndrome.

The term “probe nucleic acids and/or probe proteins” as used according to the present invention refers to nucleic acids only, proteins only and also to the simultaneous presence of nucleic acids and proteins.

According to the present invention, the term “nucleic acids” generally designates DNA and RNA of any kind and from any source. As DNA molecules cDNA can be used which can be generated from cellular or isolated mRNA.

The present invention relates to the nucleic acid chip technology, which is a very new research area, wherein the nucleic acid chips having up to several thousand spots of nucleic acid can be used. To this end, nucleic acids are immobilised on a carrier in an orderly grid pattern. The DNA or RNA to be examined (target nucleic acids or sample) is generally labelled, e.g. by using a fluorescent dye, and applied to the chip. In the case of hybridization of target nucleic acids to the probe nucleic acids bound to the carrier and having complementary sequences with the DNA or RNA to be examined a signal is detected at a corresponding position within that grid pattern, for example through a CCD camera or by a laser scanner, in a usual way.

In other words, the afore-described chip technology is based on the knowledge that the expression of a panel of molecules can be analysed simultaneously by hybridizing RNA or DNA molecules to probe nucleic acids immobilised on a carrier in an orderly grid pattern.

The present invention is described hereinafter in detail by referring to nucleic acid chips, but it should be understood that it is not limited thereto. The present invention can also be applied to protein chips where probe proteins are immobilised in an orderly grid pattern on a carrier. Proteins in the sample to be investigated can be detected by interaction with the probe proteins. This can be carried out in a usual way. The skilled artisan also knows chemicals, materials and methods to prepare protein chips and to use it accordingly, or chemicals, materials and methods suitable for protein chips can be developed in view of the details given hereinafter in connection with nucleic acid chips.

### DETAILED DESCRIPTION OF THE PREFERRED EMBODIMENT

In a preferred embodiment of the present invention, the probe nucleic acids is a gene, gene-product, splice variant of said gene and/or fragment of said gene selected from immune mediators, transcription factors, acute phase proteins, complement components, adhesion molecules, markers for cell specifity, apoptosis, housekeeping genes, or molecules associated with body response to infection. With these probe nucleic acids, the diagnosis of sepsis and sepsis related syndrome can be carried out in an advantageous way regarding the aforementioned requirements.

The term “fragment” as used herein refers to nucleic acid sequences which are shorter than the gene but still have the characteristics of said gene leading to a protein having basically the same chemical and/or physical properties, in particular functional properties, as the wild type protein.

For the diagnosis of sepsis or sepsis related syndrome, it is in particular advantageous regarding the aforementioned requirements to use a gene, gene product, splice variant of said gene and/or fragment of said gene selected from each of immune mediators, transcription factors, acute phase proteins, complement components, adhesion molecules, markers for cell specifity, apoptosis, housekeeping genes, as well as molecules associated with body response to infection and sepsis.

In a preferred embodiments of the present invention, the biochip further comprises at least one gene, gene product, splice variant of said gene and/or fragment of said gene selected from molecules of the coagulation system and/or infectious agents.

Examples of the immune mediators are growth factors, such as PDGF-α, -β, insuline like growth factor (ILGF), pro- and anti-inflammatory cytokines, such as interleukines, like IL-1 to IL-18, chemokines like platelet factor-4 (PF-4), γ-interferon induced protein 10 (IP 10), growth related proteins Gro-α, -β and -γ, eotaxin, Mip-1α, Mip-1β, RANTES, growth factor receptors, cytokine receptors, cytokine induced molecules, chemokine receptors and other cell growth-related antigens.

The transcription factors can be EGR-, NFAT- and NFkB-proteins, and members of the AP-1 and CREB protein family as well as transcription modulators.

Other acute phase proteins include C-reactive proteins and tissue factor 1 as well as cytokines, cytokine receptors, cytokine-induced molecules, chemokines, chemokine receptors, kinases, phosphatases, transcription factors and transcription modulators.

Examples of the complement components are factor H, FHL-1 and FHR encoding genes.

Markers for cell specifity include CD and adhesion molecules, T cell receptor genes and genes associated with the T cell receptor, such as CD3, CD14, CD11e/CD18, CD28, CD143 proteins, genes coding for cell specific markers such as CD4 and CD8 for T cell subtypes, CD14 for macrophages, IgM for B cells, genes of the cell cycle, genes of the eicosanoid signalling pathway and growth factor receptors.

Representatives of housekeeping genes are glucose-6-phosphate-dehydrogenase, actin and glycerinaldehyde-3-phosphate-dehydrogenase.

The additional molecules associated with body response to infection and sepsis include genes of the cell cycle, kinoses, phosphatases and transcription factors, for example the various transcripts of the human Calc genes, representing transcripts coding for procalcitonin, katatalcin N-terminal peptide, katatalcin as well as the calcitonin gene related peptide and products thereof.

Inflammation is very often caused by infectious agents like microbes. For a suitable therapy directed against the cause of the inflammation, e.g. the microbes, it is desirable to obtain as much information as possible regarding the microbes. Thus, proteins or genes of the microbes can be fixed on the carrier of the biochip used according to the present invention to obtain such information.

The biochip according to the present invention can contain one of the afore mentioned probes or any combination up to all mentioned probes. The selection of the probes can be easily determined by a person skilled in the art depending on the kind of disease to be evaluated.

The nucleic acid chip and the protein chip as described above in particular with the specific probe nucleic acids as mentioned above have a plurality of advantages in the diagnosis of sepsis and sepsis related syndrome. One advantage is that, in a very easy and fast way, the diagnosis can be carried out, in particular by using the explicitly aforementioned probe nucleic acids. The determination can be done in an accurate manner. Furthermore, the infectious agent and/or the reactions of the body caused by an infectious agent can be measured directly by detecting genes, gene fragments and/or proteins. By using the biochips as described above, the diagnosis of sepsis and sepsis related syndrome can be standardised. The pattern of expressed or defined genes is useful not only for the diagnosis, but also for the early detection, for monitoring during therapy and for the detection of the severity. In addition, the biochip used in the present invention allows a survey of the progression of the disease, the progress of sepsis and sepsis related syndrome and the success of their treatment.

For using the biochip according to the present invention, DNA or RNA to be examined can be labelled, for example by a fluorescent dye, and applied to the chip. The hybridization of the DNA or RNA to be examined to the probe molecules bound to the carrier having complementary sequences can be detected at the corresponding position within the grid through usual means, for example a CCD camera or by a laser scanner.

The biochip used according to the present invention allows an automative analysis and, furthermore, the amount of samples to be investigated can be determined quantitatively, which makes it possible to give more accurate information on the course of sepsis or sepsis related syndrome and in particular on the success of their treatment.

As already mentioned above, the probe nucleic acids and/or the probe proteins having the aforementioned specifities are immobilised on the carrier. This means that either nucleic acids only, proteins only or nucleic acids together with proteins are bound to the carrier.

According to the biochip used in the present invention, the probes are in particular immobilised on the carrier in an orderly grid pattern as usually carried out in the chip technology as described above. An orderly grid pattern includes areas on the carrier containing the probes for example as a spot. Nucleic acid chips and protein chips are generally provided with a plurality of areas on the carrier. In this case, all areas can have the same probe or the areas can have different probes, i.e. nucleic acid probes and/or protein probes.

In a preferred embodiment of the biochip used according to the present invention, the probe nucleic acids and/or probe proteins are immobilised on the carrier in predetermined areas. This means that the area on which the probes shall be immobilised, is known in advance. It also means that the location on which a specific probe is immobilised on the carrier is known before the chip is used, for example treated with the samples to be investigated. This advantageously allows a standardisation and automation of the diagnosis of sepsis and sepsis related syndrome. Furthermore, the determination is carried out in a very easy and fast way.

In a further preferred embodiment of the biochip used according to the present invention, the areas are spaced from each other. This provides, in a very advantageous manner, better and more accurate results and, furthermore, an automation and standardisation of the diagnosis of sepsis and sepsis related syndrome is possible.

Preferably, the orderly grid pattern of the probes is arranged in the form of parallel rows of the areas. Examples of such a carrier are microtiter plates, where the wells represent the areas for the probes, and which are generally arranged in parallel rows. According to this embodiment, an automation and standardisation of the determination of inflammation is achieved in an advantageous manner.

In a preferred embodiment, the carrier is a glass slide. It has favourable optical characteristics and, furthermore, it has the advantages of being solid, and not fluorescent so that it is in particular useful for the fast and accurate determination of inflammation due to these properties.

One method for the preparation of the biochip used according to the present invention shall be described hereinafter.

For the fabrication of the biochip, probes, in particular nucleic acids, can be solved in spotting solutions and thereafter they can be spotted on the carrier by a usual spotting process. As a spotting solution, saline sodium citrate (SSC) buffer is widely used. It can be suplemented with 50% dimethyl sulfoxide.

The binding of the probes, e.g. of the nucleic acids and/or the proteins, to the carrier can be achieved, either by covalent bounds or electrostatical (ionical) bounds.

For covalent binding of nucleic acids, for example compounds providing aldehyde groups are coated on the surface of the carrier. Nucleic acids contain primary amino groups which can react with the aldehyde group to form a Schiff base, i.e. a covalent bond.

For the electrostatical binding in particular of nucleic acids, use is made of the fact that they are generally negatively charged. By providing positive charges on the surface of the carrier, binding between the negatively charged nucleic acids and the positively charged surface of the carrier can be achieved by an interaction of the charges. For this purpose, glass surfaces coated with compounds providing positive charges, e.g. coated with poly-L-lysine and/or aminosilane are used. Such activated slides are well known in the art.

If necessary, cross linking of the nucleic acids with the carrier can be achieved by UV radiation.

For the use of the biochip for the diagnosis of sepsis and sepsis related syndrome, labelled mRNA, cDNA or proteins derived from humans or animals to be examined can be hybridised to the probe nucleic acids and/or probe proteins of the biochip and the hybridization pattern can be determined as already described above. Samples to be investigated can be isolated from peripheral blood cells from blood samples, in particular from venous blood, tissue samples, organs or organisms. The samples can be either used directly or can be further purified to obtain sub-groups of hematologic cell population. The blood cells can be lysed and the RNA can be isolated in the usual way. The mRNA may be further purified and cDNA can be synthesized from mRNA according to standard procedures.

Advantageously, the sample cDNA is derived from fractionated cells or from purified mRNA. Furthermore, the sample nucleic acid can be derived from PCR generated fragments. These samples show the advantage that particularly strong signals are provided.

The cDNA or mRNA used as samples can be labelled with a marker. Any substance can be used as marker which can be detected after hybridization of the samples to the probes of the biochip. Examples of such markers are fluorescent dyes. The labelled cDNA or labelled mRNA can be hybridised to the probes of the biochip in the usual way. Protein analysis can be performed by standard protein arrays technologies accordingly.

Hereinafter, the use of the biochip according to the present invention is described in more detail with reference to nucleic acids but it should be understood that the present invention is not limited to nucleic acids but can also be applied to proteins.

Cells from humans or animals to be investigated can be isolated from blood samples and peripheral blood mononuclear cells by standard procedures such as Ficoll Hypaque centrifugation or by Lymphoprep. In addition, tissues, organs or organisms such as infectious agents isolated from the patient plasma and from other biological sources can be used. The isolated cells can be used to enrich specific cell subtypes such as monocytes, T cell subtypes or B cell subtypes.

In order to purify the cells, they can be further enriched by standard protocols such as FACS sorting, magnet sorting or by lysing specific subtypes of cells. From the cells, tissues, or organisms, total RNA or fractions thereof can be isolated also by standard procedures, such as extraction by means of phenol/chloroform, RNAzol or Trizol.

The isolated RNA may be further purified to mRNA to enrich the number of coding molecules. This can be performed, for example, by oligo-dT selection by means of magnetic beads or by other procedures.

The synthesis of cDNA can also be achieved according to standard procedures. The converted cDNA may be further enriched by temperature cycling of the biological material by polymerase chain reaction using gene specific primers in combination with or in absence of oligo-dT primers.

For preparing a DNA array a carrier such as a Borofloat-33 glass slide can be coated by a thin layer of polymer useful for immobilising the DNA, such as a silane, containing reactive groups for the immobilisation of the DNA.

The hybridization of the labelled complementary RNA or a labelled complementary cDNA to the nucleic acids immobilised on the carrier can be performed according to standard protocols.

The labelling of the nucleic acids and the proteins contained in the sample to be investigated can be performed in the usual way such as incorporation reactions, for example incorporation of Cy3- or Cy5- labelled deoxynucleotides into cDNA by means of RTPCR or related reactions.

The signal detection can be performed by scanning for example with a ScanArray 2000.

The data and the quantitative expression pattern of the gene or the collection of genes can be further analysed and evaluated, for example, electronically by biocomputing using the appropriate software. For this, a computer program can be used to define a correlation of the expression of magnitude of expression of a specific gene or a group of genes with the severity of the inflammatory reactions as determined by assessing laboratory parameters or hemodynamic parameters. Such a program can be used to determine and predict the course of the severity or progression of the disease. In addition, it allows to follow the progress and effects of a therapeutic approach and intervention.

Protein arrays can be performed accordingly. However, for catching and visualisation, protein/protein-interactions can be used.

According to a preferred embodiment of the use of the present invention, the concentration of the hybridised labelled mRNA, cDNA or protein is determined. This means, in other words, that the level of nucleic acids or proteins in the sample to be investigated is determined quantitatively, which in turn allows easily to follow the course of sepsis or sepsis related syndrome, for example to see whether or not the treatment of them is successful. If it is successful, the level of expressed nucleic acids or proteins in the sample decreases, and accordingly the detected signal is lowered.

The detection of the hybridised nucleic acid or proteins can be carried out, for example, by laser scanning or CCD equipment, and multi factorial bioinformation analysis can be included to define an association of certain genes or the expression level of certain genes or proteins with the severity of that disease. Severity of acute and inflammatory reactions such as sepsis can be defined, for example, by clinical evaluation such as APACHE II scoring.

In a preferred embodiment, the biochip used according to the present invention can be used in relation to acute or chronic inflammation and, more preferably, for determining inflammation caused by sepsis or sepsis related syndromes. For detecting these diseases, the biochip according to the present invention is in particular useful and allows a fast and accurate determination.

Particularly, the severity of the inflammatory response in a particular set of patients can be determined by assessing well known laboratory and hemodynamic parameters to assess the course, the severity or progression of the disease. The analysis can be performed, for example, by relevant score systems such as APACHE II score.

The invention will be described hereinafter in more detail with reference to the example but it is to be understood that the example is intended only to illustrate the invention but not to limit it thereto.

### EXAMPLE 1

### Use of a Biochip According to the Present Invention

Whole blood was collected by venal puncture from sepsis patients who are grouped according to the APACHE II score. In addition blood was collected from healthy human volunteers. Theses samples were collected in commercially available vacutainers, which contain a solution used to stabilize RNA according to standard procedures. RNA was isolated according to the protocol of the supplier and mRNA was isolated according to standard protocols published on the worldwide web, see the publication “Microarrays.org/pdfs/polyARNAIsolation.pdf”. The cDNA was labeled by incorporating Cy3 and Cy5 labeled nucleotides by using reverse transcriptase as published on the World Wide Web, see the publication “Microarrays.org/pdfs/HumanRNALabel.pdf”.

Approximately 1000 clones were chosen from a cDNA library containing 75000 human clones (RZPD, Berlin) according to sepsis or sepsis related syndrome relevance. PCR products from these 1000 clones were spotted on poly-L-lysine coated glass slides (Telechem) by using the Omnigrid spotting machine (Genemachines).

The above-described labeled molecules to be investigated were used for hybridization to the cDNAs immobilized on the activated carrier surface as described and published on the World Wide Web at “Microarrays.org/pdfs/ArrayHybProtocol.pdf”. Washing was exactly followed according to the protocol published on the World Wide Web “Microarrays.org/pdfs/ArrayWashing.pdf”. The Genepix 4000 (Axon) scanner was used to detect the fluorescent labeled hybridized molecules according to the instruction manual. Data were compared between material obtained with sepsis patients and healthy human volunteers and the AIDA Array Evaluation software (raytest) was used for data analysis to determine up- or down regulated genes. The following data mining approaches were applied in order to determine the correlation of differential gene expression and the severity of sepsis or sepsis related syndrome. By using the above-described biochip it could be demonstrated that analysis of differential gene expression enabled the establishing of diagnoses, quantifying the severity of sepsis or sepsis related syndrome and the measurement the response to therapy.

All patents, applications, and publications referenced herein are hereby incorporated by reference herein.

1. A method of detecting a condition of sepsis or sepsis related syndrome, the method comprising: providing a chip that comprises a plurality of nucleic acid probes immobilized to the chip, wherein each of the nucleic acid probes in the plurality of nucleic acid probes are hybridizable to at least a fragment of a gene and the gene encodes a molecule associated with a body response to sepsis or infection.

2. The method of claim 1 wherein the gene is a member of the group consisting of immune mediators, transcription factors, acute phase proteins, complement components, adhesion molecules, apoptosis, housekeeping genes, markers for cell specificity, molecules of the coagulation system, and genes in an infectious agent.

3. The method of claim 1 wherein the gene is a splice variant of a gene.

4. The method of claim 1 wherein the gene is a member of the group consisting of immune mediators, transcription factors, acute phase proteins, complement components, adhesion molecules, apoptosis, housekeeping genes, and markers for cell specificity.

5. The method of claim 1 wherein the probes are immobilized to the chip in predetermined areas.

6. The method of claim 5 wherein the areas are separated from each other.

7. The method of claim 1 wherein the chip comprises a glass slide, a microtiter plate, or a nanotiter plate.

8. The method of claim 1 further comprising exposing the probes on the chip to a sample, wherein the sample comprises labeled mRNA or labeled cDNA.

9. The method of claim 8 wherein the labeled mRNA or labeled cDNA is used to quantitatively measure a concentration of mRNA, cDNA, or both mRNA and cDNA in the sample.

10. The method of claim 1 comprising examining the chip after the chip is exposed to a sample taken from the body to determine if sepsis or sepsis related syndrome is indicated in the body.

11. An assay system for diagnosis of sepsis or sepsis related syndrome in a body, wherein the assay system comprises a chip and a plurality of nucleic acid probes immobilized to the chip, wherein each of the nucleic acid probes in the plurality of nucleic acid probes are hybridizable to at least a fragment of a gene, and the gene encodes a molecule associated with a body response to sepsis or infection, wherein hybridization of the plurality of probes provides an indication concerning sepsis or sepsis related syndrome.

12. The assay system of claim 11 wherein the gene is a member of the group consisting of immune mediators, transcription factors, acute phase proteins, complement components, adhesion molecules, apoptosis, housekeeping genes, markers for cell specificity, molecules of the coagulation system, and genes in an infectious agent.

13. The assay system of claim 11 wherein the fragment of the gene is a splice variant of a gene.

14. The assay system of claim 11 wherein the gene is a member of the group consisting of immune mediators, transcription factors, acute phase proteins, complement components, adhesion molecules, apoptosis, housekeeping genes, and markers for cell specificity.

15. The assay system of claim 11 wherein the probes are immobilized to the chip in predetermined areas.

16. The assay system of claim 15 wherein the areas are separated from each other.

17. The assay system of claim 11 wherein the chip comprises a glass slide, a microtiter plate, or a nanotiter plate.

18. The assay system of claim 11 further comprising labeled mRNA or labeled cDNA hybridizable to probes on the chip.

19. The assay system of claim 18 wherein the labeled mRNA or labeled cDNA is used to quantitatively measure a concentration of mRNA, cDNA, or both mRNA and cDNA in a sample that is exposed to the chip.

20. The assay system of claim 11 comprising a sample taken from the body to determine if sepsis or sepsis related syndrome is indicated in the body after the chip is exposed to the sample.
